(19) United States
(12) Patent Application Publication
(10) Pub. No.: US 2014/0206779 A1
(43) Pub. Date: Jul. 24, 2014

(54) Methods and Systems for Converting Gaseous Hydrocarbons to Synthesis Gas

(71) Applicants: Klaus S. Lackner, Dobbs Ferry, NY (US); Eric Dahlgren, New York, NY (US); Christoph J. Meinrenken, New York, NY (US); Thomas A. Socci, Queens, NY (US)

(72) Inventors: Klaus S. Lackner, Dobbs Ferry, NY (US); Eric Dahlgren, New York, NY (US); Christoph J. Meinrenken, New York, NY (US); Thomas A. Socci, Queens, NY (US)

(21) Appl. No.: 14/001,776

(22) PCT Filed: Feb. 27, 2012
(86) PCT No.: PCT/US12/26713

§ 371 Date: Apr. 1, 2014

Related U.S. Application Data
(60) Provisional application No. 61/447,510, filed on Feb. 28, 2011.

(51) Int. Cl. C01B 3/26; C07C 1/04

(52) U.S. Cl. CPC C01B 3/26; C07C 1/04
USPC 518/703; 252/373

(57) ABSTRACT

Methods and systems for converting gaseous hydrocarbons to synthesis gas are disclosed. In some embodiments, the methods include the following: thermochemically decomposing a gaseous hydrocarbon stream in an oxygen-free environment to develop carbon and hydrogen; partially oxidizing the carbon in a hydrogen-free environment to develop carbon monoxide; mixing amounts of the carbon monoxide and the hydrogen to generate a synthesis gas; and processing the synthesis gas to generate a synthetic fuel. Some embodiments include methods for converting gaseous hydrocarbons to feedstock for producing synthesis gas including the following: partially oxidizing carbon present in a hydrogen-free part of a reactor to develop carbon monoxide, wherein oxidizing heats the reactor and the oxidizing occurs until all of the carbon is oxidized; and thermochemically decomposing a gaseous hydrocarbon stream in an oxygen-free part of the reactor to develop carbon and hydrogen, wherein the decomposing occurs until a first amount of carbon is developed.

### CROSS REFERENCE TO RELATED APPLICATION(S)

This application claims the benefit of U.S. Provisional Application No. 61/447,510, filed Feb. 28, 2011, which is incorporated by reference as if disclosed herein in its entirety.

### BACKGROUND

The U.S. has large amounts of natural gas reserves. Updated for more recently discovered fields and new extraction technologies, domestic natural gas availability is expected to significantly increase over the coming years while price projections have been corrected sharply downward (to stay below US $7 per million BTU up to 2035). It is quite possible that the US is entering a period of consistently low cost natural gas. If technology improvements maintain their momentum, even lower prices seem plausible.

While natural gas has been put to great use for carbon-lean electricity generation and heating etc., its use for the transportation sector has so far been very limited. Natural gas-powered vehicles have limited penetration potential due to safety concerns and because of the long fueling times even with fast-fill options. In addition, phasing in electric cars (powered by natural-gas derived grid electricity) will take time. Most importantly, the direct conversion from natural gas into (synthetic) gasoline or diesel, while technologically possible and performed for fringe applications, has not yet been commercially viable.

Reforming, pyrolysis, partial oxidation, autothermal reforming, fuel synthesis, and other relevant fields of art have been the subject of extensive study. However, current gas-to-liquid technologies, while available and proven, are far too capital intensive and energy inefficient to be commercially viable for the US truck and automobile fleet.

Among the fossil carbon, natural gas has proven to offer the lowest cost route to syngas. The various technologies in commercial operation today build on two chemical reactions. The most mature pathway starts with steam methane reforming. Since the syngas generated has a H2:CO ratio that is greater than the desired value of 2 the steam reforming is typically succeeded by a reverse water gas shift. A major disadvantage of this route is the endothermic nature of the two reactions, which requires additional gas to be burnt to generate the required heat. Thus, to sustain the two reactions, roughly one methane molecule has to be completely oxidized per three reacted, not counting the energy required to supply the sensible heat and elevated pressures.

A distinctly different approach is catalytic partial oxidation of the methane directly to syngas. This process delivers syngas in the desired H2:CO ratio and is slightly exothermic meaning that it requires no additional heat source like the steam methane reforming process. However, with a gas mixture containing the constituents above, highly exothermic competing reactions like should be avoided in order not to damage the reactor or deactivate the catalysts. Typical catalysts contain expensive precious metals.

### SUMMARY

Aspects of the disclosed subject matter include methods and systems for efficiently converting natural gas into drop-in liquid fuels, i.e. gasoline and diesel. Some embodiments of the disclosed subject matter include a dual-chamber reactor design to reform natural gas from existing domestic reserves or shale gas to syngas. Typical designs include a compact reactor that is energetically balanced. Other aspects of the disclosed subject matter include existing, compact Fischer-Tropsch technology to synthesize low pollutant gasoline and diesel. Typical systems will include units small enough for modular or dispersed applications.

Currently, most automobiles rely on gasoline or diesel power. Gasoline and diesel represent long-chain alkane hydrocarbons and exist as liquids. Gasoline and diesel are derived from oil reserves, many of which are located overseas and controlled by foreign governments. The United States has significant reserves of natural gas. Natural gas is similar to gasoline and diesel, however the hydrocarbon chains are shorter, therefore the fuel exists as a gas instead of a liquid and is more difficult to handle.

The Fischer-Tropsch process is a sequence of chemical reactions that turn carbon monoxide (CO) and hydrogen (H2) into liquid hydrocarbons such as gasoline and diesel. The chemical equation for the Fischer-Tropsch process is given as: nCO+(2n+1)H₂→CₙH₂ₙ₊₁+nH₂O, Syngas is a mixture of CO and H₂. Syngas is the major feedstock of the Fischer-Tropsch process. Syngas can be produced from a variety of different feedstocks.

As shown in FIG. 1, current options for converting methane to liquid hydrocarbon fuels involve complicated processes. Currently, when syngas is produced from methane, which is abundant in the US, three major chemical reactions are necessary: steam methane reforming; partial methanol oxidation; and water gas shift. As a result, the current technology for producing syngas from methane is expensive and inefficient.

Still referring to FIG. 1, aspects of the disclosed subject matter include efficient processes for turning natural gas, i.e., primarily methane, into liquid hydrocarbons such as gasoline and diesel. Processes according to the disclosed subject matter include a more direct conversion of methane to syngas and finally, to gas and diesel fuels. Processes according to the disclosed subject matter focus on the efficient production of syngas from methane, because the conversion of syngas to gasoline and diesel is well-known and scalable. Processes according to the disclosed subject matter include a novel two-step conversion of methane to syngas. One step requires energy input, i.e., is endothermic, while the other step gives off energy, i.e., is exothermic. The energy given off by the exothermic step is used to drive the completion of the endothermic step.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic diagram of methods and systems according to some embodiments of the disclosed subject matter;

FIG. 2 is a chart of a method according to some embodiments of the disclosed subject matter;

is a schematic diagram of methods and systems according to some embodiments of the disclosed subject matter;

FIG. 3 is a chart of a method according to some embodiments of the disclosed subject matter;

FIG. 4 is a schematic diagram of a reactor according to some embodiments of the disclosed subject matter;

FIG. 5 is a chart of a method according to some embodiments of the disclosed subject matter;

FIG. 6 is a diagram showing chain growth and termination of compounds produced using the Fischer-Tropsch process; and

FIG. 7 is a schematic diagram showing uses of fuels produced from syngas generated according to embodiments of the disclosed subject matter.

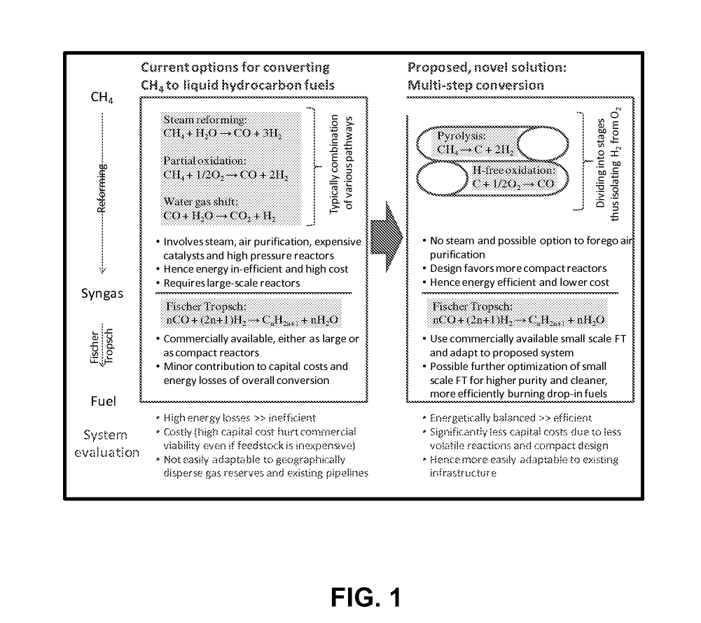

### DETAILED DESCRIPTION

Synthesis gas (CO+H₂), or syngas, for the Fischer-Tropsch process can be generated through several different pathways depending primarily on the carbon feedstock. While fossil carbon is the most economically viable feedstock today, some embodiments include the generation of syngas on a large scale in a carbon neutral way using renewable energy.

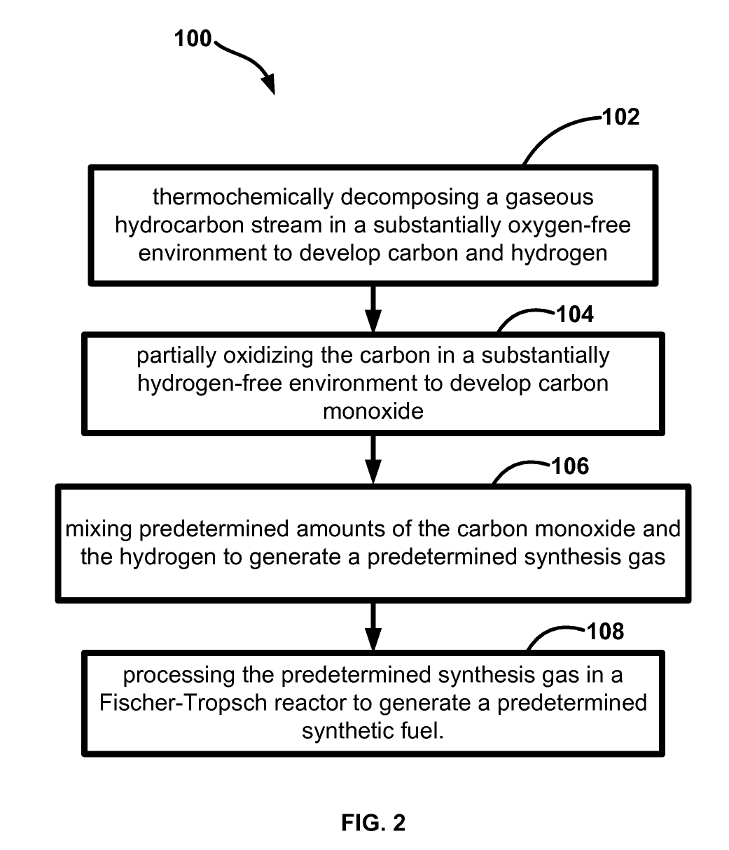

Aspects of the disclosed subject matter include methods and systems for converting gaseous hydrocarbons to synthesis gas. Referring now to FIG. 2, some embodiments include a method 100 for converting gaseous hydrocarbons to synthesis gas. At 102, thermochemically decomposing a gaseous hydrocarbon stream in a substantially oxygen-free environment to develop carbon and hydrogen; gaseous hydrocarbons include methane decomposing is performed at about 700 to about 100 degrees Celsius. decomposing is performed in the presence of ceramic catalysts At 104, partially oxidizing said carbon in a substantially hydrogen-free environment to develop carbon monoxide; At 106, mixing predetermined amounts of said carbon monoxide and said hydrogen to generate a predetermined synthesis gas. At 108, processing said predetermined synthesis gas in a Fischer-Tropsch reactor to generate a predetermined synthetic fuel.

Some embodiments of the disclosed subject matter include an overall partial oxidation reaction similar to above but carried out in two steps: 1) pyrolysis of a gaseous hydrocarbon, e.g., for example, but not limited to, methane, natural gas, etc.; followed by 2) partial oxidation of the solid carbon in a hydrogen free environment. For example, assuming the gas is methane since this is the primary component of natural gas, the reactions would be as follows:

CH₄═C+2H₂ΔH1100K=77 kJ/mole; and

C+0.5O₂═CO,ΔH1100K=−98 kJ/mole.

Methane decomposes in its elements above 500° C. but in order to reach 99% conversion the temperature should be at least 1100° C. when basically only the elements are stable At temperatures above 700° C., partial oxidation of carbon is thermodynamically preferential to complete oxidation and the equilibrium of the Boudouard reaction

2CO⇄C+CO2

is shifted to the left. Thus, thermodynamics gives us a lower operating temperature of 1100° C. in order to reach 99% conversion. The process could be operated below 1100° C. (but above 700° C.) but then in order to reduce waste the unconverted hydrocarbons should be recycled.

The desirable feature of ceramic catalysts used in pyrolysis is a lowering of the operating temperature. Not only does this entail a lower energy profile of the entire syngas production process it also lessen the propensity of NOx formation which places less stringent requirement on the purity of the oxygen stream. If the stability of the intended ceramic catalysts proves dissatisfying under the alternating conditions of a reducing and oxidizing environment, other metal oxides, e.g., aluminum oxide, can be used.

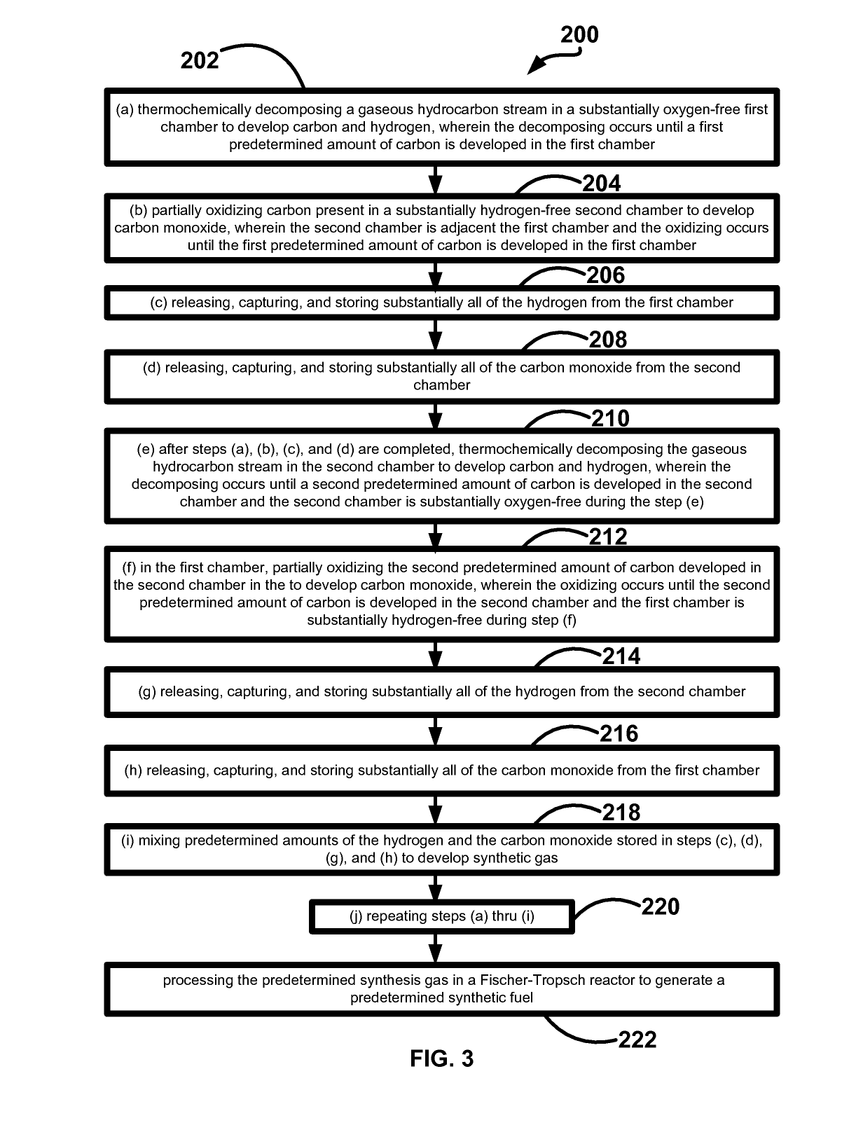

Referring now to FIG. 3, some embodiments include a method 200 for converting gaseous hydrocarbons to synthesis gas. At 202, a gaseous hydrocarbon stream, e.g., natural gas/methane, is thermochemically decomposed in a substantially oxygen-free first chamber to develop carbon and hydrogen. The gaseous hydrocarbon stream is provided to the first chamber and the decomposing occurs until a first predetermined amount of carbon is developed in the first chamber. At 204, carbon present in a substantially hydrogen-free second chamber partially oxidized to develop carbon monoxide. Oxygen is fed to the second chamber during step 204. The second chamber is adjacent the first chamber and the oxidizing occurs until the first predetermined amount of carbon is developed in the first chamber. The oxidizing is exothermic thereby generating heat that is transferred to an adjacent chamber. The decomposing and oxidizing occur substantially simultaneously. At 206, substantially all of the hydrogen in the first chamber is released from the first chamber, captured, and stored. At 208, substantially all of the carbon monoxide in the second chamber is released from the second chamber, captured, and stored. At 210, after steps 202, 204, 206, and 208 are completed, the gaseous hydrocarbon stream in the second chamber is thermochemically decomposed to develop carbon and hydrogen. The decomposing occurs until a second predetermined amount of carbon is developed in the second chamber. The second chamber is substantially oxygen-free during the step 210. Decomposing in step 210 includes feeding methane to the second chamber. At 212, in the first chamber, the second predetermined amount of carbon developed in the second chamber is partially oxidized to develop carbon monoxide. The oxidizing occurs until the second predetermined amount of carbon is developed in the second chamber. The first chamber is substantially hydrogen-free during step 212. Oxidizing in step 212 is exothermic thereby generating heat that is transferred to an adjacent chamber. Oxidizing in step 212 includes feeding oxygen to the first chamber. At 214, substantially all of the hydrogen in the second chamber is released from the second chamber, captured, and stored. At 216, substantially all of the carbon monoxide in the first chamber is released from the first chamber, captured, and stored. At 218, predetermined amounts of the hydrogen and the carbon monoxide stored in previous steps are mixed to develop synthetic gas. At 220, the previous steps are repeated to produce additional synthetic gas. At 222, the synthesis gas produced is processed in a Fischer-Tropsch reactor to generate a predetermined synthetic fuel. In some embodiments, method 200 is practiced in a reactor including a plurality of the first and second chambers arranged in a honeycomb-like structure. In some embodiments, the reactor is fabricated from one or more of aluminum oxide, silicon oxide, and magnesium oxide.

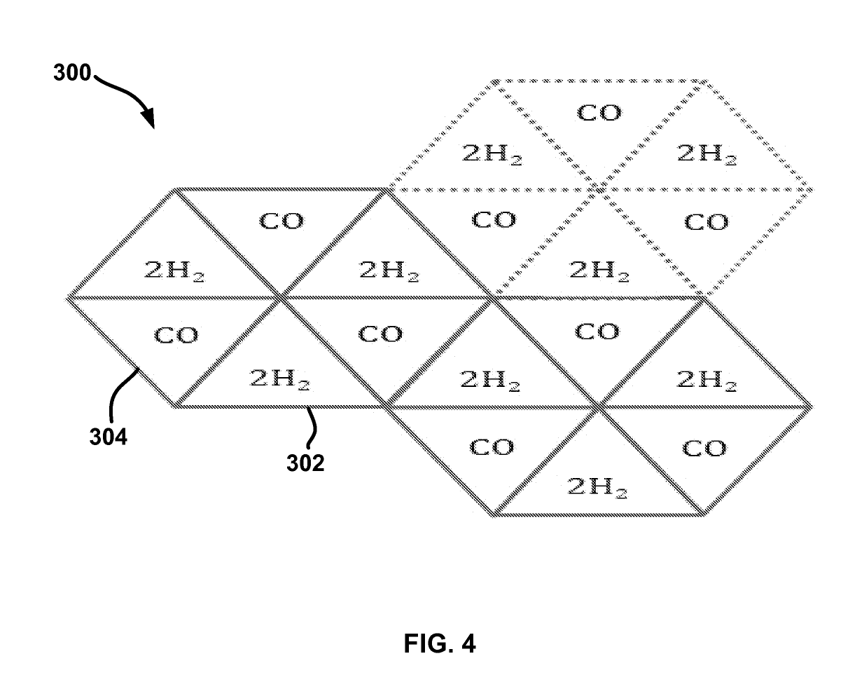

Method 200 is typically practiced using a dual-stream reactor. Referring now to FIG. 4, in some embodiments, two feed streams, a gaseous hydrocarbon stream, for example but not limited to methane, and an oxygen containing mixture, for example, but not limited to, pure oxygen or air, are fed simultaneously to mutually adjacent volumes in a honeycomb structured reactor 300. That is, all neighboring volumes 302 to the oxygen containing stream 304 are fed gaseous hydrocarbon and vice versa. This structure allows for optimal heat transfer since the exothermic partial oxidation of the solid carbon is co-located with the endothermic pyrolysis. In this way, a continuous deposition of carbon and hydrogen evolution occurs in half the overall reactor volume while carbon monoxide is produced in the other half. After all the solid carbon has been oxidized, the flows are alternated such that the volumes that were fed gaseous hydrocarbons now are fed the oxygen containing mixture and vice versa.

Depending on the application and configuration, the two outgoing streams are put in thermal connection with the incoming streams to allow for heat transfer between the incoming and outgoing streams for the dual-stream reactor. If not, complete conversion of the hydrocarbon stream is performed and a separation of the product hydrogen and the unconverted hydrocarbons can be performed after this heat exchange, for example, but not limited to using membrane separation. The produced hydrogen can then be mixed with the carbon monoxide in the other stream in order to form the desired syngas.

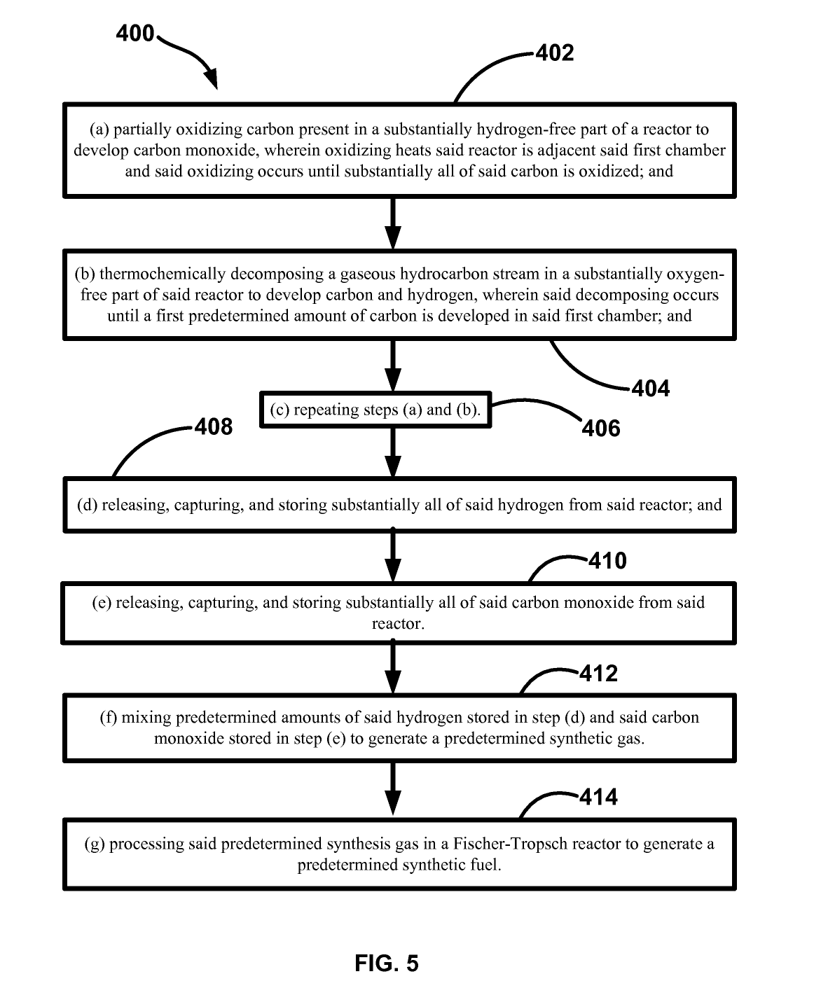

Referring now to FIG. 5, some embodiments include a method 400 for converting gaseous hydrocarbons, e.g., natural gas/methane to feedstock for producing synthetic gas. At 402, carbon present in a substantially hydrogen-free part of a reactor is partially oxidized to develop carbon monoxide. Oxidizing heats the reactor and the oxidizing occurs until substantially all of the carbon is oxidized. At 404, a gaseous hydrocarbon stream is thermochemically decomposed in a substantially oxygen-free part of the reactor to develop carbon and hydrogen The decomposing occurs until a first predetermined amount of carbon is developed. At 406, steps 402 and 404 are repeated. At 408, substantially all of the hydrogen contained in the reactor is released from the reactor, captured, and stored. At 410, substantially all of the carbon monoxide contained in the reactor is released, captured, and stored. At 412, predetermined amounts of the hydrogen stored are mixed with predetermined amounts of the carbon monoxide stored to generate a predetermined synthetic gas. At 414, the predetermined synthesis gas is processed in a Fischer-Tropsch reactor to generate a predetermined synthetic fuel.

In use, method 400 is typically practiced in a single-stream reactor. In comparison with dual-stream reactor 300 described above where the exothermic and endothermic reactions occur simultaneously, embodiments including a single-stream reactor have a sequential operation. Assuming the reactor volume contains previously deposited carbon, the oxygen is fed to the volume. The heat of the exothermic oxidation reaction is adsorbed by the walls of the volume. After the carbon (or other respective material) has reacted to carbon monoxide (or equivalent) gaseous hydrocarbon is fed into the same volume and the heat stored in the reactor walls is transferred to the gas and carbon (or equivalent) is again deposited. The geometry of the reactor volume and flow speed is adjusted to achieve a plug flow and hence now back mixing of the different streams inside the reactor. The two streams can be allowed to mix after the reactor if close to complete conversion of the hydrocarbon gases has been accomplished. If not, a separation of the streams succeeding the reactor can be invoked.

In some embodiments, multiple single stream reactors are positioned closely together so as to achieve optimal heat conservation. In some embodiments, the outgoing streams are put in thermal connection with the incoming streams to allow for heat transfer between the incoming and outgoing streams for the single-stream reactor.

As mentioned above, in some embodiments, the reactors are fabricated from materials including for example, but not limited to, ceramics such as aluminum oxide, silicon oxide, magnesium oxide, carbon in various forms, and any other material that can withstand both oxidizing and reducing environments at elevated temperatures as well as serve as catalysts for the reaction.

The disclosed subject matter is informed by a history of catalytic fuel synthesis that operated under assumptions of monolithic scale-up to large units. First developed in 1923 and in ever-expanding commercial use today, the Fischer-Tropsch process consists of the hydrogenation of adsorbed CO to form CH₂ “monomers” for stepwise oligomerization on catalyst surfaces. The source of the reactants is a synthesis gas of carbon monoxide and hydrogen, which are adsorbed by the catalyst and undergo the following overall exothermic reaction:

CO+2H₂→CH₂—+H₂O+165 kJ.

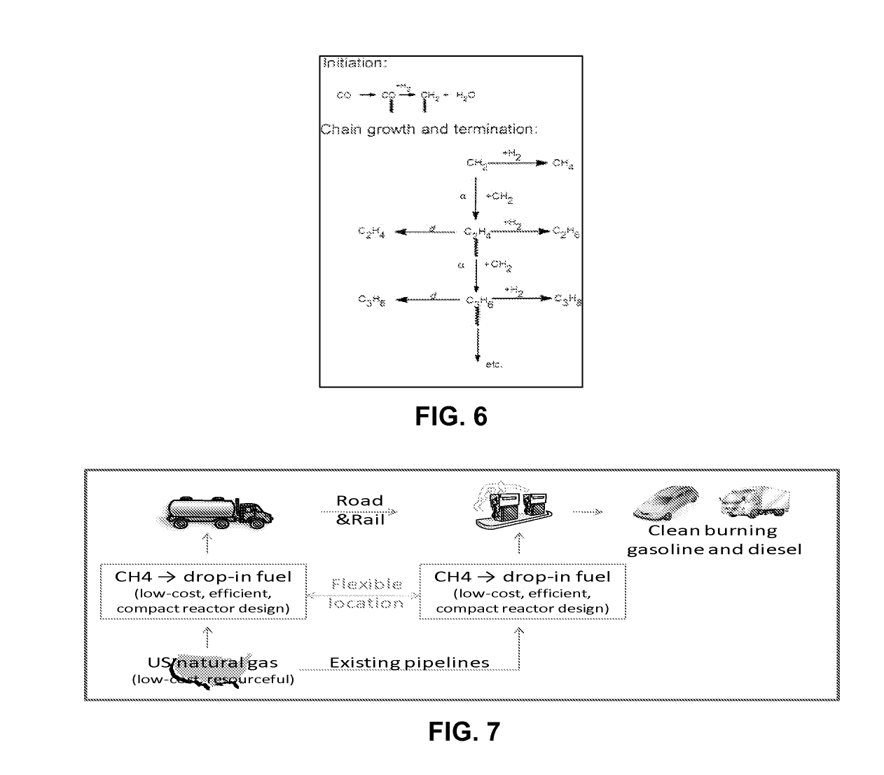

At each stage, the newly formed adsorbed hydrocarbon can desorb, hydrogenate, or continue chain growth with another monomer. Referring now to FIG. 6, desorption or chain growth proceeds according to some probability parameter (here d, α, respectively). The result is a suite of hydrocarbon paraffin waxes and olefins of varying chain length and industrial applicability, particularly gasoline and diesel. Synthetic fuels produced by this process are sulfur-free and nitrogen-free, and are therefore chemically cleaner than those produced from crude oil.

Historically, research and development of the process assumed singular, large scale structures, embracing and discarding various reactor designs under the assumption that the ability to scale-up to larger and larger reactor units was a critical criterion for reactor choice. The exothermic nature of the reaction requires that scale-up designs have sufficient heat exchange to maintain an exothermic reaction environment; large cooling areas are required to shed this heat. The issue of pressure drop with increasing bed length is also a scale-up concern, as is observed in the long, narrow reaction chambers of multi-tubular fixed bed reactors, or in re-circulating reactors that employ multiple passes of gaseous streams to maximize the conversion of syngas to hydrocarbon product. Maintaining these process conditions is of critical importance for the Fischer-Tropsch synthesis, as increased temperature favors elective methane formation (an undesired product), deposition of catalyst-damaging carbon, and reduced chain length of products. Thus, the most prolific reactor design in commercial use today is the slurry bubble column reactor, which exhibits on large scales the requisite low pressure drop, excellent heat transfer characteristics for stable reactor temperatures, no diffusion limitations, continuous online refreshment of catalyst particles, and relatively simple construction at low investment capital cost. As of 2007, the slurry bubble column and the multi-tubular reactor design were the only two Fischer-Tropsch models with significant market share.

The Fischer-Tropsch reaction as a fuel synthesis option takes the advantages of smaller units one step further, given the sensitivity of this process to temperature, pressure, input gas stoichiometric ratios of carbon and hydrogen, catalyst type, and promoters. Existing reactors separate and recycle the output streams back into their own input stream to maximize conversion, but a network of smaller scale reactors allows output streams to be refined in terms of these parameters and redirected to different small-scale units whose conditions are optimized for the products of choice. These smaller units host reactions of shorter residence time, but are operated by process automation that can make decisions in real-time redirecting the small-unit tail gas to optimal reaction conditions. The secondary reactions that occur in a Fischer-Tropsch reactor, as recycled olefins have been demonstrated to be catalytically reabsorbed for further transformation and synthesis, are managed in systems according to the disclosed subject matter. Enhanced control of reactants allows more selective control of products.

Some embodiments of the disclosed subject matter include a fuel synthesis unit having sub-units such as for example but not limited to the following:

1. A merger sub-unit that combines input streams from either an internal syngas unit or the outputs of other units;

2. A catalysis chamber, in which the fuel synthesis reaction takes place via Fischer-Tropsch or another similar catalytic fuel synthesis;

3. A separator sub-unit which separates the output stream on the basis of weight or on the basis of boiling point or on some other basis in order to selectively distribute the throughput to other units in the network;

4. A cracking sub-unit in which the products of the fuel synthesis unit can be further selectively broken down into more desirable hydrocarbons or into hydrocarbons that can be re-fed into downstream units or into another profile of hydrocarbons;

5. A second separation may also or instead occur after a cracking sub-unit has already processed the throughput; and

6. Additional sub-units not specified above.

In some embodiments, a unit includes multiple inputs, either from external syngas preparation or internal syngas preparation, or from the selectively separated and cracked outputs of other units. Furthermore, in some embodiments, there are multiple outputs from a unit, e.g., including an output from a cracker that consists of the desirable hydrocarbons or hydrocarbons of one profile that have bypassed a cracker via earlier separation. In some embodiments, the preceding sub-units are internal to a standard module of the fuel synthesis network or instead incorporated into a network as varieties of units.

One aspect of the disclosed subject matter is the separation in time and/or in space of the pertinent two or more reactions described above, e.g., the dissociation of CH₄ into C and H₂ versus the oxidation of C to CO. As disclosed with respect to method 200, the separation in space is achieved by executing the reactions in multiple separate reaction volumes, for example, but not limited to, a collection of pipes, reactors, cubes, or other separations such as, but not limited to, magnetic containment that contain the reactants and control the intended chemical or physical reactions by any method known to one of ordinary skills in the art, such as, for example, but not limited to, supplying or retracting specific other reactants, catalysts, temperatures/heat, electromagnetic or radioactive radiation, pressures, or vibrations that set of the desired reactions. As disclosed with respect to method 400, a separation in space is achieved in a single containment design, e.g., but not limited to, a long, thin pipe, by controlling the reaction separately in specific parts of the single container with any method known to one of ordinary skills in the art, such as, for example, but not limited to, supplying or retracting specific other reactants, catalysts, temperatures/heat, electromagnetic or radioactive radiation, pressures, or vibrations that set of the desired reactions. Some embodiments include a single container design as described above, e.g., single chamber.

In some embodiments, a separation in time is achieved by providing parts of a container or specific ones of multiple containers only at specific times with the control mechanisms of the desired reaction via any method known to one of ordinary skills in the art, such as, for example, but not limited to, supplying or retracting specific other reactants, catalysts, temperatures/heat, electromagnetic or radioactive radiation, pressures, or vibrations that set of the desired reactions.

Another aspect of the disclosed subject matter is the controlled transfer of heat within or between the one or various container/chambers. In embodiments described above, this heat transfer is achieved via conduction. Other embodiments may employ for the purposes of the heat transfer any method known to one of ordinary skills in the art, such as, for example, but not limited to, radiation or convection. In yet other embodiments, the controlled heat transfer extends to the subsequent reactions of the overall desired reaction, such as, for example, but not limited to Fischer-Tropsch. In such an embodiment, the compact Fischer-Tropsch reactors are co-localized and tightly integrated with the syngas production to leverage the exothermic nature of the Fischer-Tropsch to balance entropic losses during the 2-step syngas production, e.g., but not limited to, heat exchange with incoming gas streams to the multi-step reforming reactor.

Still another aspect of the disclosed subject matter is selecting the particular geometry of the one or several containers such that they provide the optimum of above mechanisms for described separation in time and/or space, heat transfer, and reaction control, via, for example, but not limited to, specific geometric arrangements of the reaction volume structure that also allows required access to the structure for supplying the ingoing substances and reaction products, e.g., via pipes, ducts, holes, etc. As discussed above with respect to reactor 300, some embodiments include a particular reactor design and geometry, e.g., honeycomb of triangles. However, some embodiments include spheres, squares, ovals, rolled up tubes, etc. The geometry may also be movable such that above mentioned separation and reaction control mechanisms are achieved via a deliberate change in the overall geometry during the reaction, e.g., rotating tubes, re-arranging various reaction volumes relative to each other, or changing the shape of the contained volume itself, for example, but not limited to, a change in the magnetic containment, that achieves changes in for example, but not limited to heat conductance, heat radiation, electro-magnetic radiation, etc.

In some embodiments of the disclosed subject matter, the intentional separation of reactions in space and or time, and/or the controlled supply of the reaction controls, e.g., such as gases, heat, electromagnetisms, pressure, etc., are applied not only to the syngas reforming of equivalent reaction step but also to the subsequent step such as, for example, but not limited to Fischer Tropsch such that an overall integrated system of the various above the distinct but interrelated parts and aspects of the invention is achieved to yield yet improved usability.

Methods and systems according to the disclosed subject matter offer benefits and advantages over known technology. Since the proposed gas-to-liquid process takes as its feedstock a syngas derived from domestic natural gas, designs according to the disclosed subject matter present a direct 1:1 displacement/reduction of petroleum use in the transportation sector: every gallon of synthetic gasoline or diesel produced by the novel technology saves a gallon that would otherwise have the be obtained via oil refining.

Designs according to the disclosed subject matter helps to significantly reduce non-CO₂ tail pipe pollutants, e.g., VOCs, etc., incrementally reduce CO₂, e.g., more efficient gasoline, and indirectly displace soot and other pollutants typically associated with petroleum refineries.

Designs according to the disclosed subject matter also afford a flexibility that suggests the opportunity to move away from gasoline and diesel as the only transportation fuels. These convenient products of oil refining are the status quo among transportation energy carriers, but in a post-oil world the assumption that these are the most easily produced carbon chains is no longer true. The Fischer-Tropsch process itself is evidence of the fungible nature of carbon resources, and is readily able to produce whatever flavor of hydrocarbon is de rigueur. For example, it has been shown in processes that can generate 90% gasoline in the product suite that DME generation in the presence of certain catalysts is highly effective in promoting the Fischer-Tropsch synthesis reactions. However, DME could in turn become the desired product and its production from methanol synthesis units can be either inhibited where methanol is the product of choice or encouraged via dehydration. DME has been demonstrated to be an efficient choice of turbine fuel, a competitive automotive fuel, functional as residential fuel for heating and cooking, non-toxic and non-carcinogenic.

In addition to the obvious advantages of reduced foreign oil dependence and compatibility with existing fuel distribution and vehicles/engines, designs according to the disclosed subject matter offer the further advantage of low tailpipe pollution, e.g., organic compounds, as well as reduced pollution from petroleum refining. Furthermore, the technology complements ongoing and future improvements in vehicle propulsion and engine technology, e.g., hybrids, advanced lubricants, low aerosol combustion, lean NOx catalyst development, etc.

In addition to cost and energy efficiency, designs according to the disclosed subject matter reduce pollution associated with refining, e.g., soot, etc., and the resulting synthetic gasoline, diesel, and optional kerosene fuels can be fine-tuned to burn more efficiently and cleaner. Complementing future further improvement in engine technology, this will reduce green house gases (GHG) as well as soot and volatile organic compounds, e.g., VOC, e.g. aerosols, etc.

Designs according to the disclosed subject matter offers the additional advantage of a relatively compact, low capital reactor design. As shown in FIG. 7, this makes designs according to the disclosed subject matter ideally suited to be gradually phased in and integrated with the existing, geographically diverse infrastructure of natural gas reserves, gas pipelines, liquid fuel trucking, gas stations, and transportation modes in the U.S.

Designs according to the disclosed subject matter will aid the U.S. in extending the use of inexpensive gas to the transportation sector without requiring technological changes to current or future vehicle technology, e.g., compatible with plug-in hybrid cars and other GHG reduction efforts, without impact on agriculture practices/land use, and yet without reliance on petroleum.

Designs according to the disclosed subject matter will supply infrastructure-ready liquid fuels derived from domestic natural gas resources. The fossil-derived liquid fuels produced by the Fischer-Tropsch process are combustible in present-day transportation and industrial infrastructure. In fact, synthetic fuels are superior to those derived from petroleum refining, as they contain far fewer impurities and can be tailored to higher octane ratings by virtue of the ability to fine-tune the product spectrum as one aim of designs according to the disclosed subject matter.

Compared to steam methane reforming, methods and systems according to the disclosed subject matter are superior for producing syngas. Separating the reaction in two steps, 1) pyrolysis of methane to carbon and hydrogen and 2) partial oxidation of solid carbon in a hydrogen free environment, provides benefits over known steam methane reforming methods.

Working with a solid phase in reforming processes poses obvious mass and heat transfer issues when operating on large physical unit scales. Methods and systems according to the disclosed subject matter that include small individual reactor volumes gives a more favorable surface area to volume ratio and circumvent not only the difficulties facing partial methane oxidation but in other chemical processes as well.

Methods and systems according to the disclosed subject matter are close to energy-balanced, therefore efficiency losses are much reduced. Furthermore, the dual-chamber syngas production favors more compact, less capital intensive reactor design. Some embodiments of the disclosed subject matter do not require 100% oxygen purification.

A second important aspect facilitating the commercial viability of designs according to the disclosed subject matter is that it does not require high, upfront capital expenditure for building large, durable reactors. The proposed design will enable smaller, more compact reactors that can be built cheaper and amortize sooner. This way, the technology can sidestep the inherent risk of long term natural gas versus oil price projections, thus offering a much more favorable risk profile than current GTL technology.

Commercial viability of designs according to the disclosed subject matter is aided by two additional, crucial aspects. Other than the novel GTL plants themselves, which can be phased in over time and co-located with either (shale) gas extraction or at the end of existing gas pipelines, the technology does not require any other infrastructure changes. Existing fuel infrastructure, e.g., pipelines, fuel trains/trucks, gas stations, can continue to be used, as can existing vehicle and engine technologies. However, the technology does open the door for more advanced fuels in the future. Secondly, the alternative fuel has no impact on agriculture practices/land use.

The following are additional advantages of methods and systems according to the disclosed subject matter:

US natural gas: extends the benefits of plentiful and relatively inexpensive US natural gas reserves to the transportation sector;

Petroleum displacement: Making synthetic gasoline and diesel without any use of petroleum reduces foreign oil dependency as well as pollutants (e.g., soot) associated with oil refineries;

Drop-in fuel compatible with current infrastructure: The resulting fuels can be used in existing cars (gasoline) and trucks (diesel), and, if desired, the technology could be extended to aircraft (kerosene). The technology is further compatible with existing fuel distribution networks (trucks, pipelines, gas stations), as well as with emerging fleets of parallel, series, and plug-in hybrid vehicle technology;

Reduced tail-pipe pollutants: Fischer Tropsch-derived synthetic fuels are of higher purity and thus naturally low polluting, significantly reducing (non CO₂) tail pipe exhausts such as volatile organic compounds (VOC, e.g., aerosols, etc.);

Reduction of carbon dioxide emissions: Although the carbon in the synthetic fuels will be natural-gas derived, i.e. fossil, designs according to the disclosed subject matter will not altogether replace fossil carbon dioxide emission. Still, some reduction will be facilitated, via three mechanisms: 1) The synthetic fuels do not require any change in engine technology, and are thus ideally suited to complement ongoing fuel reduction efforts such as hybrids (see above); 2) Advanced implementations of the proposed compact Fischer Tropsch enable advanced designer fuels that achieve higher efficiencies (more miles per gallon) in future optimized internal combustion engines; and 3) The synthetic fuel technology facilitates the future reduction of heavy fuel oil use (currently a by-product from gasoline refining). For example, some of the GHG-intensive fuel oil could be replaced with natural gas heating.

In addition to displacing petroleum, the methods and systems according to the disclosed subject matter reduce pollution associated with refining (soot, etc.), and the resulting synthetic gasoline, diesel (and optional kerosene) fuels can be fine-tuned to burn more efficiently and cleaner. Complementing future further improvement in engine technology, this will reduce GHG as well as soot and volatile organic compounds (VOC, e.g. aerosols, etc.).

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

1. A method for converting gaseous hydrocarbons to synthesis gas, said method comprising:
thermochemically decomposing a gaseous hydrocarbon stream in a substantially oxygen-free environment to develop carbon and hydrogen;
partially oxidizing said carbon in a substantially hydrogen-free environment to develop carbon monoxide; and
mixing predetermined amounts of said carbon monoxide and said hydrogen to generate a predetermined synthesis gas.

2. The method according to claim 1, wherein said gaseous hydrocarbons include methane.

3. The method according to claim 1, further comprising:
processing said predetermined synthesis gas in a Fischer-Tropsch reactor to generate a predetermined synthetic fuel.

4. The method according to claim 1, wherein said decomposing is performed at about 700 to about 1000 degrees Celsius.

5. The method according to claim 1, wherein said decomposing is performed in the presence of ceramic catalysts.

6. A method for converting gaseous hydrocarbons to synthesis gas, said method comprising:
(a) thermochemically decomposing a gaseous hydrocarbon stream in a substantially oxygen-free first chamber to develop carbon and hydrogen, wherein said decomposing occurs until a first predetermined amount of carbon is developed in said first chamber;
(b) partially oxidizing carbon present in a substantially hydrogen-free second chamber to develop carbon monoxide, wherein said second chamber is adjacent said first chamber and said oxidizing occurs until said first predetermined amount of carbon is developed in said first chamber;
(c) releasing, capturing, and storing substantially all of said hydrogen from said first chamber;
(d) releasing, capturing, and storing substantially all of said carbon monoxide from said second chamber;
(e) after steps (a), (b), (c), and (d) are completed, thermochemically decomposing said gaseous hydrocarbon stream in said second chamber to develop carbon and hydrogen, wherein said decomposing occurs until a second predetermined amount of carbon is developed in said second chamber and said second chamber is substantially oxygen-free during said step (e);
(f) in said first chamber, partially oxidizing said second predetermined amount of carbon developed in said second chamber in said to develop carbon monoxide, wherein said oxidizing occurs until said second predetermined amount of carbon is developed in said second chamber and said first chamber is substantially hydrogen-free during step (f);
(g) releasing, capturing, and storing substantially all of said hydrogen from said second chamber;
(h) releasing, capturing, and storing substantially all of said carbon monoxide from said first chamber; and
(i) mixing predetermined amounts of said hydrogen and said carbon monoxide stored in steps (c), (d), (g), and (h) to develop synthetic gas.

7. The method according to claim 6, further comprising:
(j) repeating steps (a) thru (i).

8. The method according to claim 6, wherein said gaseous hydrocarbons include methane.

9. The method according to claim 6, further comprising:
processing said predetermined synthesis gas in a Fischer-Tropsch reactor to generate a predetermined synthetic fuel.

10. The method according to claim 6, wherein oxidizing in steps (b) and (f) is exothermic thereby generating heat that is transferred to an adjacent chamber.

11. The method according to claim 6, further comprising:
providing a reactor including a plurality of said first and second chambers arranged in a honeycomb-like structure.

12. The method according to claim 11, wherein said reactor is fabricated from one or more of aluminum oxide, silicon oxide, and magnesium oxide.

13. The method according to claim 6, wherein oxidizing in steps (b) and (f) includes feeding oxygen to said respective second and first chambers.

14. The method according to claim 6, wherein decomposing in steps (a) and (e) includes feeding methane to said respective second and first chambers.

15. The method according to claim 6, wherein decomposing and oxidizing occur substantially simultaneously.

16. A method for converting gaseous hydrocarbons to feedstock for producing synthetic gas, said method comprising:
(a) partially oxidizing carbon present in a substantially hydrogen-free part of a reactor to develop carbon monoxide, wherein oxidizing heats said reactor and said oxidizing occurs until substantially all of said carbon is oxidized;
(b) thermochemically decomposing a gaseous hydrocarbon stream in a substantially oxygen-free part of said reactor to develop carbon and hydrogen, wherein said decomposing occurs until a first predetermined amount of carbon is developed; and
(c) repeating steps (a) and (b).

17. The method according to claim 16, further comprising:
(d) releasing, capturing, and storing substantially all of said hydrogen from said reactor; and
(e) releasing, capturing, and storing substantially all of said carbon monoxide from said reactor.

18. The method according to claim 17, further comprising:
(f) mixing predetermined amounts of said hydrogen stored in step (d) and said carbon monoxide stored in step (e) to generate a predetermined synthetic gas.

19. The method according to claim 16, wherein said gaseous hydrocarbons include methane.

20. The method according to claim 16, further comprising:
(g) said predetermined synthesis gas in a Fischer-Tropsch reactor to generate a predetermined synthetic fuel.
